(19) United States
(12) Patent Application Publication
(10) Pub. No.: US 2005/0123490 A1
(43) Pub. Date: Jun. 9, 2005

(54) Composition and method for prevention and treatment of dental caries

(76) Inventor: Kazue Yamagishi, Tokyo (JP)

Correspondence Address: PALMER & DODGE, LLP;PAULA CAMPBELL EVANS, 111 HUNTINGTON AVENUE, BOSTON, MA, 02199, US

(21) Appl. No.: 11/001,467

(22) Filed: Dec. 1, 2004

Related U.S. Application Data
(60) Provisional application No. 60/526,890, filed on Dec. 4, 2003.

(51) Int. Cl. A61K 7/16; A61K 7/20

(52) U.S. Cl. 424/53; 433/217.1

(57) ABSTRACT

The present invention relates to compositions and methods for prevention and treatment of dental caries. A dental paste is produced from a silicate containing hydroxyapatite (SiHAp) powder, an aqueous solution of hydrogen peroxide and an aqueous solution of phosphoric acid. The dental paste is applied to an enamel of a tooth, dissolves a portion of the enamel and integrates with the enamel of the tooth to produce a synthetic enamel. The dental paste crystallizes by integrating with a chemically incomplete portion of the tooth. The synthetic enamel comprises a chemical composition and a crystal structure approximately the same as the hydroxyapatite of a tooth prior to treatment with the dental paste.

### RELATED APPLICATIONS

This application claims priority to U.S. Provisional Application Ser. No. 60/526,890, filed on Dec. 4, 2003, and the entirety of this application is incorporated herein by reference.

### FIELD OF THE INVENTION

The present invention relates to dental treatments, and more particularly to a composition and method for the prevention and treatment of dental caries.

### BACKGROUND OF THE INVENTION

The proper care, maintenance and repair of teeth is of utmost concern for people all over the world. From the earliest ages, people are taught dental maintenance steps including teeth brushing, teeth flossing and regular dental checkups. The prevention of dental caries is important for dental health. Almost 20 percent of children between the ages of 2 and 4 have had dental caries, and by the age of 17, about four out of five people have had at least one carious lesion (i.e., cavity) or restoration (filling). Over two-thirds of adults between 35 and 44 years old have lost at least one permanent tooth and about one-half of people 75 years or older have had root caries affecting at least one tooth. Glock, Martha; Horowitz, Alice M.; Canto, Maria T., compilers, Diagnosis and Management of Dental Caries. Bethesda (Md.): National Library of Medicine; 2001 February, Current Bibliographies in Medicine (CBM), no. 2001-1 (available at www.nlm.nih.gov/pubs/cbm/dental_caries.html).

Dental caries are common oral and infectious diseases that result in the destruction of the tooth structure by the invasion of acid forming bacteria that are found in dental plaque, an intraoral biofilm. Scientific evidence has shown that the initiation of dental caries requires a relatively high proportion of mutans streptococci bacteria which is found within dental plaque. This bacteria adheres well to the surface of the tooth and produces higher amounts of acid from sugars than other bacteria types. Dental caries often appears as a white chalky area on the enamel of the tooth, that later softens and progresses to the breakdown of the tooth structure. Depending upon the severity of the dental caries, the disease can lead to the loss of tooth minerals, and the extraction of the tooth. Mineral loss is inhibited by the availability of fluoride in the mouth, as the fluoride promotes remineralization of the tooth structure which leads to rebuilt and stronger outer layers of the tooth.

Dental caries are dependent on dietary sucrose that change both the thickness and the chemical nature of the plaque. A diet with a high proportion of sucrose increases the risk of dental caries. When the plaque bacteria come into contact with various foods or liquids containing simple sugars, the bacteria metabolize the sugars and make organic acids as a metabolic by-product. If the acids are not buffered by saliva, the acids dissolve the surface of the apatite crystals of adjacent tooth structure, a process known as demineralization. In thick gel-like plaque, the pH falls with a couple of seconds of contact with dietary sugars. When the pH is neutral, the same crystals can regrow, using calcium, phosphate, and fluoride from saliva causing remineralization. Dental carie disease begins when demineralization outweighs remineralization.

Dental caries can be modified by fluoride. The minerals of enamel, cementum and dentin are a highly substituted calcium phosphate salt called apatite. While the apatite of newly formed teeth is relatively soluble, rich in carbonate and low in fluoride, demineralization and remineralization in a fluoride rich environment creates apatite of lower carbonate, higher fluoride and lower solubility. Fluorides present in foods, drinks, dentrifices, oral rinses and filling materials can reduce the solubility of teeth and reduce the risk of dental caries.

The prior art has not provided an adequate solution to preventing dental caries from forming or for treating dental caries. Adherence to proper dental care, including tooth brushing, flossing and regular dental checkups, may prevent the formation of dental caries. Prior art devices and methods have provided mechanical solutions for treating dental caries. The prior art mechanical devices and methods of treating dental caries are invasive and may require local anesthesia. Prior art devices use low speed and high speed handpieces in drilling procedures to remove dental caries, followed by a subsequent placement of restorative materials to replace the void. Prior art treatments using organic resin, polymer and inorganic dental cement have not been successfully used to replace early lesion of dental caries. The organic resins, polymers and inorganic dental cements do not bury the dental carie for a sufficient time without deep excavation of the affected dental carie. Because an affected part of the dental carie is microscopic in the early stages, organic resins, polymers and inorganic dental cements lie on the tooth and are easily removed over time. The difference in the rates of thermal expansion between the enamel and the organic resin, polymer and inorganic dental cements prevent adequate adhesion between the enamel and the organic resin, polymer and inorganic dental cements.

U.S. Pat. No. 5,952,399 to Rentsch discloses a polymerisable dental material and use of apatite fillers in the dental material. The Rentsch material has variably adjustable transparency, good polishability, high strength and capacity to release and absorb ions into and from a biological environment. The Rentsch material is a dental material filler that only provides an aesthetic solution for the teeth.

U.S. Pat. No. 5,972,311 to Blackshear discloses a method of preventing the formation of dental caries and other oral lesions and removing sugars from an oral cavity. The Blackshear method includes an oral hygiene composition that includes at least one carbohydrate-binding protein, and most preferably a lectin or other sucrose or galactose/glucose binding protein. The Blackshear method is used to remove sugars and is not effective for preventing non-sugar related sources of dental caries. In addition, the Blackshear method does not integrate into the tooth to prevent subsequent caries or produce a hydroxyapatite synthetic enamel.

U.S. Pat. No. 6,454,566 to Lynch et al. discloses an apparatus for the treatment of dental caries that includes a source of oxidizing gas and a handpiece for delivering the gas to a tooth. A cup attached to a handpiece of the Lynch et al. device is provided for receiving the gas and exposing a selected area of the tooth having the dental carie to the gas, the cup having a resilient edge for engaging and sealing the tooth. The Lynch et al. cup does not adequately seal the tooth to prevent escape of the gas into the mouth of a patient, thereby exposing the patient to health risks. The Lynch et al. device can remove the existing dental carie without preventing future dental caries and leaves a void in the surface of the enamel of the tooth.

### SUMMARY OF THE INVENTION

The present invention provides compositions and methods for the prevention and treatment of dental caries. The compositions of the present invention offer several advantages over conventional compositions used to prevent and treat dental caries of a tooth including integrating with a chemically incomplete portion of the tooth to crystallize the chemically incomplete portion of the tooth and replace the dental carie; and integrating with an enamel of the tooth to form a synthetic enamel.

The present invention is a dental paste to treat a dental carie of a tooth comprising: a silicate containing hydroxyapatite powder; an aqueous solution of hydrogen peroxide; and an aqueous solution of phosphoric acid.

The present invention is a method of treating a dental carie of a tooth including: (a) applying a dental paste comprising a silicate containing hydroxyapatite powder mixed with an aqueous solution of hydrogen peroxide and an aqueous solution of phosphoric acid to the tooth having the dental carie; and (b) washing an excess amount of the dental paste from the tooth. Steps (a) and (b) may be repeated several times until the dental carie is treated. The method may further include applying a second dental paste comprising a fluorized apatite powder mixed with an aqueous solution of hydrogen peroxide and an aqueous solution of phosphoric acid to the tooth having the dental carie and washing an excess amount of the second dental paste from the tooth.

The present invention is a method of preventing formation of a dental carie including: (a) applying a dental paste comprising a silicate containing hydroxyapatite powder mixed with an aqueous solution of hydrogen peroxide and an aqueous solution of phosphoric acid to the tooth and (b) washing an excess amount of the dental paste from the tooth. The method may further include applying a second dental paste comprising a fluorized apatite powder mixed with an aqueous solution of hydrogen peroxide and an aqueous solution of phosphoric acid to the tooth and washing an excess amount of the second dental paste from the tooth.

The present invention is a method of making a dental paste to prevent and treat a dental carie comprising: mixing an aqueous solution of phosphoric acid-sodium silicate and a calcium phosphate; filtering a precipitate of a mixture of the aqueous solution of phosphoric acid-sodium silicate and the calcium phosphate; reducing the precipitate to a plurality of small particles and passing the plurality of small particles through a sleeve to yield a silicate containing hydroxyapatite powder; and mixing the silicate containing hydroxyapatite powder with an aqueous solution of hydrogen peroxide and an aqueous solution of phosphoric acid.

The present invention is a method of whitening a tooth that includes: applying a dental paste comprising a silicate containing hydroxyapatite powder mixed with an aqueous solution of hydrogen peroxide and an aqueous solution of phosphoric acid to the tooth; and washing an excess amount of the dental paste from the tooth. The method may further include applying a second dental paste comprising a fluorized apatite powder mixed with an aqueous solution of hydrogen peroxide and an aqueous solution of phosphoric acid to the tooth and washing an excess amount of the second dental paste from the tooth.

In addition to offering all of the advantages discussed above, the compositions and methods of the present invention are safe, simple to prepare, effective and require a short duration and painless procedure. These and other advantages of the present invention will be obvious through the embodiments described hereinafter.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a cross section view of a tooth.

FIG. 2 is a top view of a tooth with a dental carie at an approximate center portion of the tooth.

FIG. 3 is a top view of a tooth treated using the dental paste containing FHAp of the present invention producing a synthetic enamel that replaces a dental carie.

FIG. 4A is a scanning electron microscope image of a portion of a polished cross section of a tooth, including a synthetic enamel, after application of dental paste containing FHAp of the present invention.

FIG. 4B is a back scattered scanning electron microscope image of a portion of a polished cross section of a tooth, including a synthetic enamel, after application of the dental paste containing FHAp of the present invention.

FIG. 4C is a secondary scanning electron microscope image of an inner structure of a portion of a polished tooth, including a synthetic enamel, after application of the dental paste containing FHAp of the present invention.

FIG. 4D is a high magnification secondary scanning electron microscope image of a synthetic enamel after application of the dental paste containing FHAp of the present invention.

FIG. 5A is a wide scan x-ray photoelectron spectroscopy spectrum of a synthetic enamel of a tooth after application of the dental paste containing FHAp of the present invention.

FIG. 5B is a x-ray diffraction (XRD) pattern of a synthetic enamel of a tooth after application of the dental paste containing FHAp of the present invention.

FIG. 6A is an atomic force microscopy image of a polished surface of an enamel of a tooth before treatment with the dental paste containing FHAp of the present invention.

FIG. 6B is an atomic force microscopy image of a polished surface of a tooth after application of the dental paste containing FHAp of the present invention to an enamel of the tooth.

FIG. 6C is an atomic force microscopy image of a surface of a tooth covered with a plurality of particles of the dental paste containing FHAp of the present invention after application of the FHAp dental paste.

FIG. 7A is a low magnification scanning electron microscope image of a portion of a polished cross section of a tooth, including a synthetic enamel, after application of the dental paste containing SiHAp of the present invention.

FIG. 7B is a high magnification scanning electron microscope image of a synthetic enamel after application of the dental paste containing SiHAp of the present invention.

FIG. 8 is a low magnification scanning electron microscope image of a portion of a polished cross section of a tooth, including a synthetic enamel, after application of the dental paste containing FHAp of the present invention.

FIG. 9A is a low magnification transmission electron microscope image of a thin tooth section showing the synthetic enamel (R) and the enamel (E) after treatment with the dental paste containing FHAp of the present invention.

FIG. 9B is a magnified transmission electron microscope image of a thin tooth section showing the synthetic enamel after treatment with the dental paste containing FHAp of the present invention.

FIG. 9C is a magnified transmission electron microscope image of a thin tooth section showing the enamel after treatment with the dental paste containing FHAp of the present invention.

FIG. 9D is a magnified transmission electron microscope image of a thin tooth section showing the interface between the synthetic enamel and the enamel after treatment with the dental paste containing FHAp of the present invention.

FIG. 9E is a high-resolution transmission electron microscope image of a thin tooth section showing the crystal structure in the synthetic enamel after treatment with the dental paste containing FHAp of the present invention.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides compositions and methods for the prevention and treatment of dental caries. An embodiment of the present invention provides a dental paste comprising a fluorized apatite (FHAp) powder, an aqueous solution of hydrogen peroxide and an aqueous solution of phosphoric acid. In an embodiment of the present invention, the fluorized apatite powder is comprised of a calcium deficient hydroxyapatite powder and an aqueous solution of sodium fluoride. Another embodiment of the present invention provides a dental paste comprising silicate containing hydroxyapatite (SiHAp) powder, an aqueous solution of hydrogen peroxide and an aqueous solution of phosphoric acid.

For a tooth with a dental carie, a dental paste of the present invention, either alone or in combination, is applied to a dental carie in an enamel of a tooth and then integrates with the enamel of the tooth to form a synthetic enamel. The integration of the enamel and the synthetic enamel occurs within the depth of the natural enamel of the tooth. The integration of the synthetic enamel and the enamel of the tooth should not extend beyond the dentin and enamel junction, although it may extend beyond the dentin and enamel junction. The dental paste integrates with the existing chemically incomplete portion of the enamel of the tooth by an inorganic crystallization process which completes the chemically and physically incomplete portion of the enamel of the tooth to replace the dental carie. For a healthy tooth, the dental paste of the present invention, either alone or in combination, is applied to the tooth, integrates with the enamel of the tooth to form a synthetic enamel and completes the incomplete natural enamel structure.

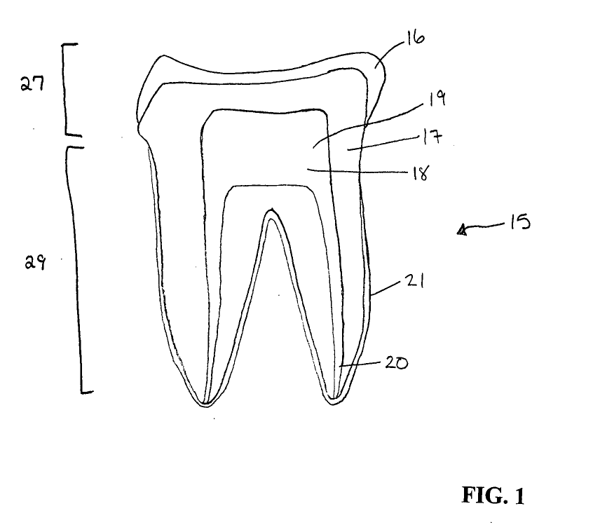

FIG. 1 shows a vertical cross section of a tooth 15. The tooth 15 comprises an exposed crown 27 and a root 29. The crown 27 is usually at least partially covered by an enamel 16, an outer layer of a hard substance similar to bone. Beneath the enamel 16 is a dentin 17, an intermediate layer of material which is also similar to bone but not as hard as the enamel 16. The crown 27 comprises the enamel 16 and a portion of an outermost section of the dentin 17 located proximal to the enamel 16. The dentin 17 surrounds an inner pulp cavity 18 comprised of a pulp 19. Blood vessels and nerves reach the inner pulp cavity 18 through a channel called a root canal 20 that penetrates the root 29. The root 29 is the lower portion of the tooth 15 that comprises the root canal 20, and at least a portion of the inner pulp cavity 18. A cementum 21, a thin coating, covers the root 29 of the tooth 15.

The enamel 16 is calcified bone that serves as a protective layer of the tooth 15. The enamel 16 is comprised of a plurality of small enamel rods or prisms which form the framework of the tooth 15. The enamel 16 is translucent and can vary in color from yellowish to grayish white based upon variations in thickness, translucent properties, and the quality of the crystal structure and surface stains of the enamel 16. The enamel 16 is comprised of about 96% inorganic materials, about 1% organic materials and about 3% water. The main inorganic material components of the enamel 16 are calcium and phosphorus as a hydroxyapatite.

The dentin 17 is a light yellow and porous substance that is more radiolucent than the enamel 16. The dentin 17 comprises the largest portion of the tooth 15 and is harder than bone, but softer than the enamel 16. The dentin 17 carries sensations such as temperature and pain to the pulp 19. The dentin 17 comprises about 70% inorganic material and about 30% organic material and water. The main inorganic material components of the dentin are calcium and phosphorus as a hydroxyapatite.

The pulp 19 is the only soft tissue of the tooth 15 and makes the tooth 15 a living tissue. The pulp 19 contains blood vessels, nerves and other cells and supplies nutrients to the tooth 15 and the nerve endings of the pulp transmit sensations such as pain and temperature. The pulp 19 responds to irritation, either by forming reparative secondary dentin or by becoming inflamed.

The cementum 21 is a bonelike tissue that is light yellow in color and slightly lighter in color than the dentin 17. The cementum 21 is comprised of about 55% organic material and about 45% inorganic material. The main inorganic material components are calcium salts.

FIG. 2 shows a top view of the tooth 15 comprising a dental carie 44, shown as a line at an approximate center portion of the enamel 16 of the tooth 15. FIG. 2 shows the top of the tooth 15 before a treatment of the tooth 15 by a dental paste 36 containing FHAp of the present invention. The dental carie 44, also known as a cavity, is a soft, decayed area in the tooth 15 that can progress from the enamel 16 to the dentin 17 and ultimately the pulp 19 of the tooth 15. Bacteria present in the mouth convert all foods, and especially sugars and starches into acids. Bacteria, acid, food debris and saliva combine in the mouth to form a sticky substance called plaque that adheres to the teeth. Plaque that is not removed from the teeth 15 mineralizes into calculus or tartar. The acids in the plaque dissolve at least a portion of the enamel 16 of the tooth 15, creating the dental carie 44.

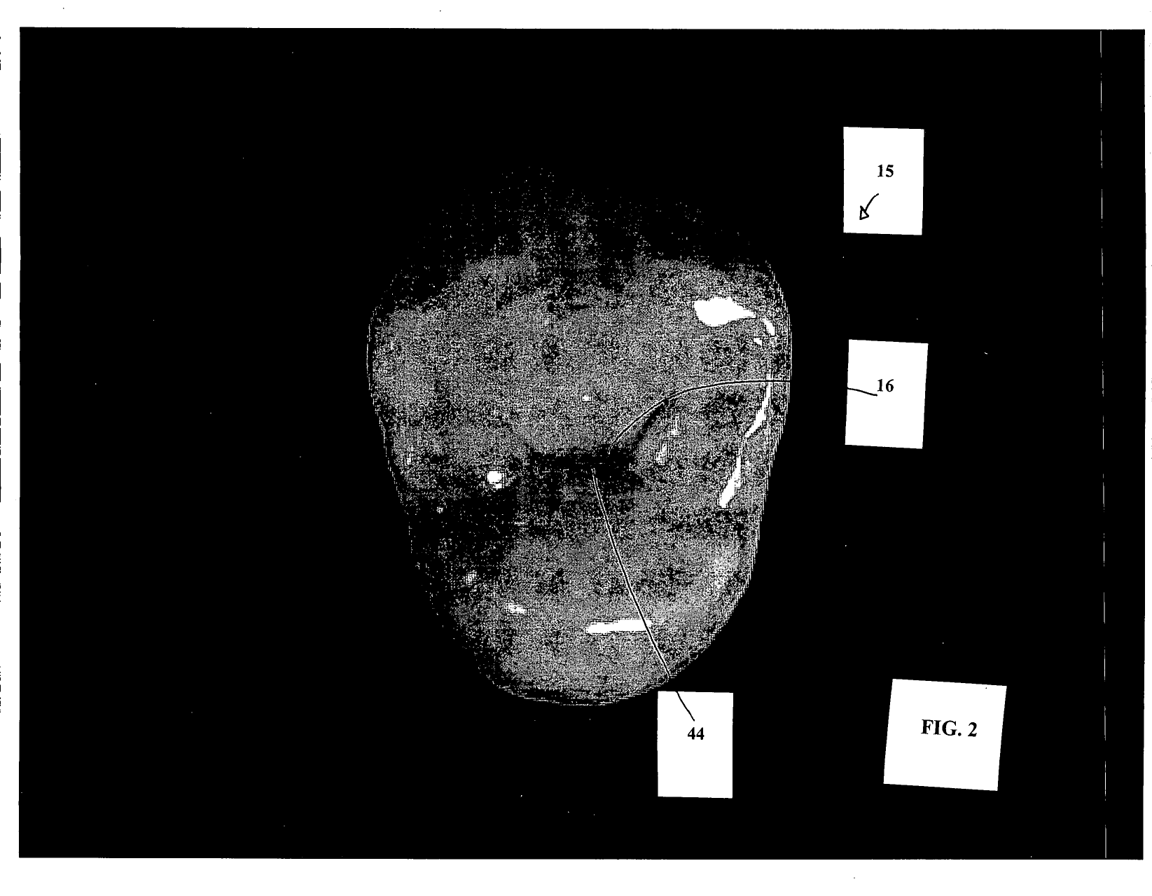

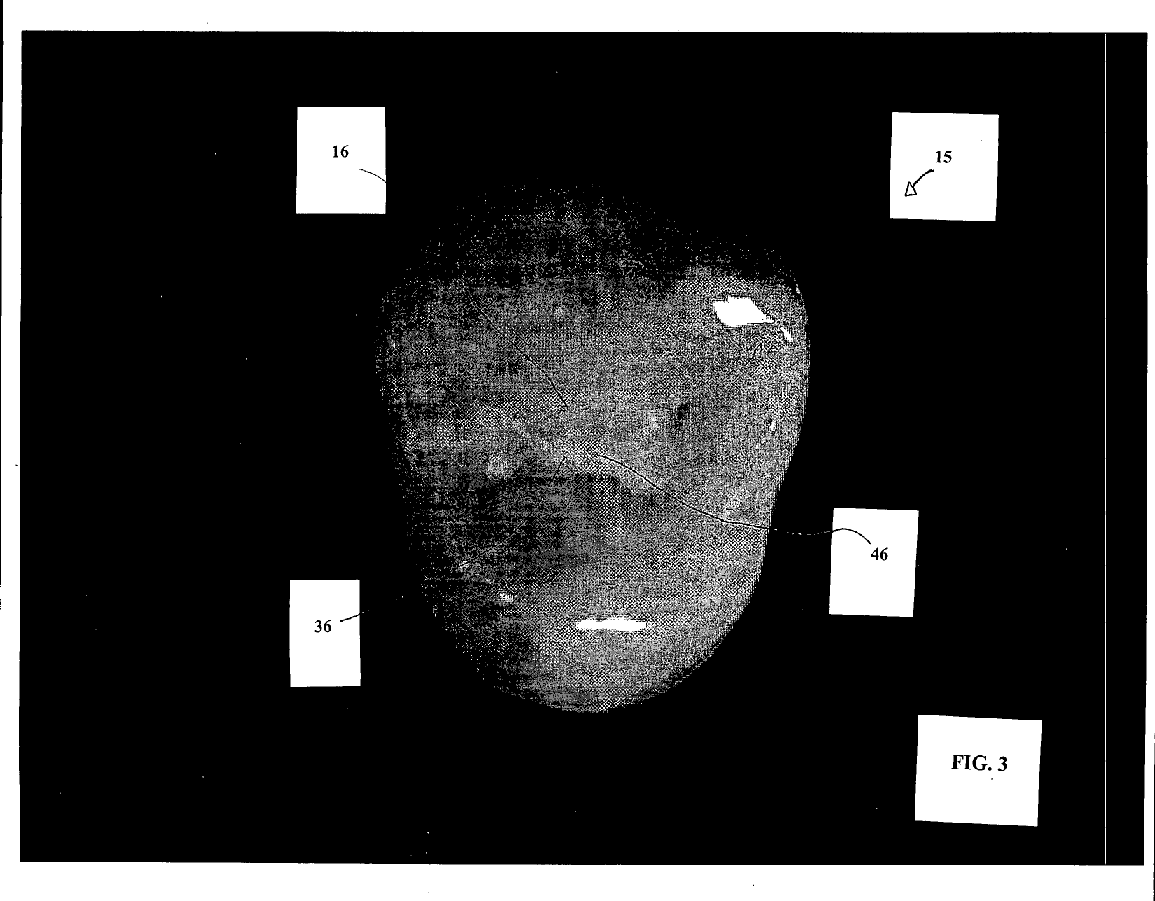

FIG. 3 shows a top view of the tooth 15 after treatment of the dental carie 44 through an application of the dental paste 36 containing FHAp of the present invention that integrates with the enamel 16 of the tooth 15 to form a synthetic enamel 46. As shown in FIG. 3, the dental carie 44, shown as the line in the center portion of the tooth of FIG. 2, is removed and replaced with the synthetic enamel 46.

The present invention also prevents the formation of the dental carie 44 in a manner similar to the treatment of the dental carie 44. The chemistry and mechanism for preventing the formation of a dental carie 44 and the treatment of a dental carie 44 are similar. In one embodiment of the present invention, for a healthy tooth 15, the dental paste 36 containing FHAp is applied to the tooth, integrates with the enamel of the tooth 15 to form a synthetic enamel 46, and completes the incomplete natural enamel structure. In another embodiment of the present invention, the dental paste 38 containing SiHAp is applied to the tooth, integrates with the enamel of the tooth 15 to form a synthetic enamel 46, and completes the incomplete natural enamel structure. In another embodiment of the present invention, both the dental paste 36 containing FHAp and the dental paste 38 containing SiHAp are applied to the tooth, integrate with the enamel of the tooth 15 to form a synthetic enamel 46 and completes the incomplete natural enamel structure.

FIG. 2 and FIG. 3 can be used to discuss the prevention of the formation of a dental carie 44. A tooth 15 having an incomplete enamel structure is susceptible to the formation of the dental carie 44. The formation of a dental carie 44 on a healthy tooth 15 involves a process whereby the healthy tooth 15 has a susceptibility to a dental carie 44 and, if not properly treated, progresses to the formation of a dental carie 44. A dental paste of the present invention, either alone or in combination, integrates with the incomplete enamel structure of a tooth 15 to form the synthetic enamel 46 and mechanically and chemically completes the enamel structure of the tooth 15 to prevent the formation of the dental carie 44. The extent of integration is governed by the depth of the natural enamel 16 of the tooth 15. The dental paste completes the apatite of the enamel 16 to prevent the formation of the dental carie 44.

In an embodiment of the present invention, the dental paste is comprised of a fluorized apatite (FHAp) powder, an aqueous solution of hydrogen peroxide and an aqueous solution of phosphoric acid. In an embodiment of the present invention, the fluorized apatite powder is comprised of a calcium phosphate and an aqueous solution of sodium fluoride. Calcium phosphates, especially hydroxyapatite, have physiochemical properties similar to those of the enamel 16. In an embodiment of the present invention, the calcium phosphate is a calcium deficient hydroxyapatite powder. In another embodiment of the present invention, the calcium phosphate is a hydroxyapatite. In another embodiment of the present invention, the calcium phosphate is a hydroxyapatite and a calcium deficient hydroxyapatite. In another embodiment of the present invention, the calcium of the calcium phosphate may be substituted partially by strontium, barium, cadmium, lead, copper, sodium, potassium and others. Preparation of the dental paste 36 containing FHAp that prevents and treats the dental carie 44 consists of several steps.

The hydroxyapatite includes calcium phosphate materials with various chemical compositions. In one embodiment of the present invention, the chemical composition is Ca₁₀(PO₄)₆(OH)₂. In another embodiment of the present invention, the hydroxyapatite is a calcium deficient hydroxyapatite with a calcium to phosphorus molar ratio between about 0.5 and about 1.67. In another embodiment of the present invention, the hydroxyapatite is a calcium excess hydroxyapatite with a calcium to phosphorus molar ratio larger than about 1.67. In another embodiment of the present invention, the hydroxyapatite is a carbonated apatite with either the whole or part of the PO₄³⁻ ions and/or OH⁻ ion are substituted by the CO₃²⁻ ion. In another embodiment of the present invention, the hydroxyapatite is a fluorapatite and a chlorapatite in which either the whole or part of the OH⁻ ion are substituted by F− ions and/or Cl− ions. In another embodiment of the present invention, the hydroxyapatite is a cation substituted hydroxyapatite in which part of the calcium (Ca) ion is substituted by a metal such as magnesium, zinc, strontium and others. In another embodiment of the present invention, the hydroxyapatite is a combination of the calcium deficient hydroxyapatite, the fluorapatite and chlorapatite.

In another embodiment of the present invention, the fluorized apatite powder is comprised of the hydroxyapatite and other thermodynamically metastable calcium phosphates. A thermodynamically metastable material transforms to the hydroxyapatite with time under wet conditions such as in solution or in gel like paste, especially when the hydroxyapatite coexists. In one embodiment of the present invention, the thermodynamically metastable calcium phosphate is amorphous calcium phosphate having a representative chemical composition of Ca₉(PO₄)₆*nH₂O. In another embodiment of the present invention, the thermodynamically metastable calcium phosphate is selected from the group consisting of octacalcium phosphate (Ca₈(HPO₄)₂(PO₄)₄*5H₂O, dicalcium phosphate dihydrate (CaHPO₄*2H₂O), dicalcium phosphate anhydrous CaHPO₄, tricalcium phosphate (whitlockite) including α and β phases (Ca₃(PO₄)₂, β-Ca₃(PO₄)₂, tetracalcium phosphate (Ca₄(PO₄)₂O), monocalcium phosphate monohydrate Ca(H₂PO₄)₂*H₂O and monocalcium phosphate anhydrous (Ca(H₂PO₄)₂). Those skilled in the art will recognize there are other thermodynamically metastable calcium phosphates known in the art that are within the spirit and scope of the present invention. In an embodiment of the present invention, about 1 gram (g) of calcium deficient hydroxyapatite powder (calcium to phosphorus (Ca/P) ratio of about 1.64) (available from UBE Material, Ltd., Japan) is added to about 100 milliliters (ml) of an about 200 millimolar (mM) concentration aqueous solution of sodium fluoride. After the mixture of the calcium deficient hydroxyapatite powder and the sodium fluoride is stirred for about one hour at about 60° C., the mixture is filtered and a precipitate from the mixture of the calcium deficient hydroxyapatite powder and the sodium fluoride is obtained.

In an embodiment of the present invention, the mixture of the calcium deficient hydroxyapatite powder and the sodium fluoride is stirred with a Bioshaker. The precipitate is then washed with water and dried at about 110° C. for about 24 hours, yielding a fluorized apatite powder. In an embodiment of the present invention, the fluorized apatite powder is a fluoride treated calcium deficient hydroxyapatite powder.

In the process of the formation of the fluorized apatite (FHAp) powder, the OH of the calcium deficient hydroxyapatite powder (Ca₁₀(PO₂)₆(OH₂) is substituted with F⁻ for stability, allowing the calcium deficient hydroxyapatite powder to convert into the stable fluorized apatite powder (Ca₁₀(PO₄)₆F₂). The fluoride content of the fluorized apatite powder can be about 0.3 weight to weight percent (w/w %). In the present invention, the fluoride content of the fluorized apatite powder preferably is between about 0.01 to about 1.0 weight to weight percent (w/w %). The fluorized apatite powder is crushed and passed through a 100 mesh sleeve (aperture size about 150 microns). About 2 ml of a mix of an about 35% aqueous solution of hydrogen peroxide and an about 85% aqueous solution of phosphoric acid is added to and mixed with about 1.5 g of the fluorized apatite powder to yield the dental paste 36 containing FHAp of the present invention. Those skilled in the art will recognize that various aqueous solutions and powders known in the art can be used with the present invention and other amounts and concentrations of the aqueous solutions and powders in addition to other processing conditions known in the art are within the spirit and scope of the present invention.

The phosphoric acids used to yield the dental paste 36 containing FHAp includes, but are not limited to, metaphosphoric acid, pyrophosphoric acid, ortho-phosphoric acid and triphosphate. In a preferred embodiment of the present invention, the phosphoric acid content of the dental paste 36 containing FHAp is between about 1.0 to about 20 w/w %. Those skilled in the art will recognize other acids known in the art can be used with the present invention and are within the spirit and scope of the present invention.

After the dental paste 36 containing FHAp is mixed, the dental paste 36 containing FHAp is applied to the enamel 16 of the tooth 15 where the dental paste 36 containing FHAp integrates with the enamel 16 of the tooth 15. In integrating with the enamel 16, the dental paste 36 containing FHAp fills in a in a lattice of the hydroxyapatite compound comprising the enamel 16. The dental paste 36 containing FHAp crystallizes with a chemically incomplete portion of the enamel 16 of the tooth, forming fluorized apatite crystals of the synthetic enamel 46. In a preferred embodiment of the present invention, the ingredients used to make the dental paste 36 containing FHAp are mixed prior to use, as the dental paste 36 containing FHAp will undergo gradual loss of activity. About fifteen minutes after application of the dental paste 36 containing FHAp to the enamel 16, the excess dental paste 36 containing FHAp that has not integrated with the enamel 16 is removed by washing the tooth 15 with a washing medium. In a preferred embodiment of the present invention, the washing medium is purified water. Those skilled in the art will recognize that the dental paste can remain on the tooth for various lengths of time and be within the spirit and scope of the present invention.

After application of the dental paste 36 containing FHAp, the dental paste 36 containing FHAp dissolves a portion of the enamel 16 of the tooth 15. The dental paste 36 containing FHAp is comprised of a strong acid having a pH of about 1-2 from the addition of the aqueous solutions of hydrogen peroxide and phosphoric acid. The phosphoric acid is the primary contributor to the low pH of the dental paste 36 containing FHAp. The dental paste 36 containing FHAp dissolves the enamel 16 on a microscopic level to allow for the integration of the dental paste 36 containing FHAp within the enamel 16. The dental paste 36 containing FHAp is removed after a specified time to dissolve a specified amount of the enamel 16 to produce the synthetic enamel 46 through integration of the dental paste 36 containing FHAp. After dissolution of a portion of the enamel 16, there is a recrystallization as the fluorized apatite crystals of the synthetic enamel 46 are formed. The extent of integration is governed by the depth of the natural enamel 16 of the tooth 15.

In an embodiment of the present invention, the dental paste 36 containing FHAp is applied to the enamel 16 of the tooth 15 several times. In an embodiment of the present invention, the dental paste 36 containing FHAp is applied to the enamel 16 of the tooth 15 from about two times to about six times. In an embodiment of the present invention, the dental paste 36 containing FHAp is applied to the enamel 16 of the tooth 15 about three times. Each application of the dental paste 36 containing FHAp to the tooth 15 increases the thickness of the synthetic enamel 46, allowing for additional integration of the dental paste 36 containing FHAp into the enamel 16 of the tooth 15. After each application of the dental paste 36 containing FHAp to the tooth 15, the excess dental paste 36 containing FHAp is removed by washing the tooth 15 with the washing medium. In an alternative embodiment of the present invention, a high and/or low speed handpiece is used in addition to the application of the dental paste 36 containing FHAp. Those skilled in the art will recognize that any number of applications of the dental paste to the tooth are within the spirit and scope of the present invention. Those skilled in the art will also recognize that the dental paste 36 containing FHAp can be removed from the tooth 15 in many ways known in the art and be within the spirit and scope of the present invention.

For a tooth showing a susceptibility to the formation of a dental carie 44, the dental paste 36 containing FHAp integrates with the incomplete enamel structure to form the synthetic enamel 46. The dental paste 36 containing FHAp mechanically and chemically completes the enamel structure of the tooth 15, completing the apatite of the enamel 16 to prevent the dental carie 44 from forming.

The dental paste 36 containing FHAp of the present invention treats the dental carie 44 and replaces the dental carie 44 with the synthetic enamel 46. Those skilled in the art will recognize the dental paste 36 containing FHAp can be used to treat dental caries 44 in various stages ranging from a stage where there is no dental carie 44 to stages where the dental carie 44 is deep within the tooth 15 and be within the spirit and scope of the present invention.

For the tooth having a dental carie 44, the dental paste 36 containing FHAp is used to treat the dental carie 44 existing in the enamel 16 of the tooth 15. In another embodiment of the present invention, the dental paste 36 containing FHAp is used to treat the dental carie 44 that has advanced into the dentin 17 of the tooth 15. In another embodiment of the present invention, the dental paste 36 containing FHAp can be used as a cement. Those skilled in the art will recognize the dental paste 36 containing FHAp can be used to treat dental caries 44 that have progressed into various portions of the tooth 15 and are within the spirit and scope of the present invention.

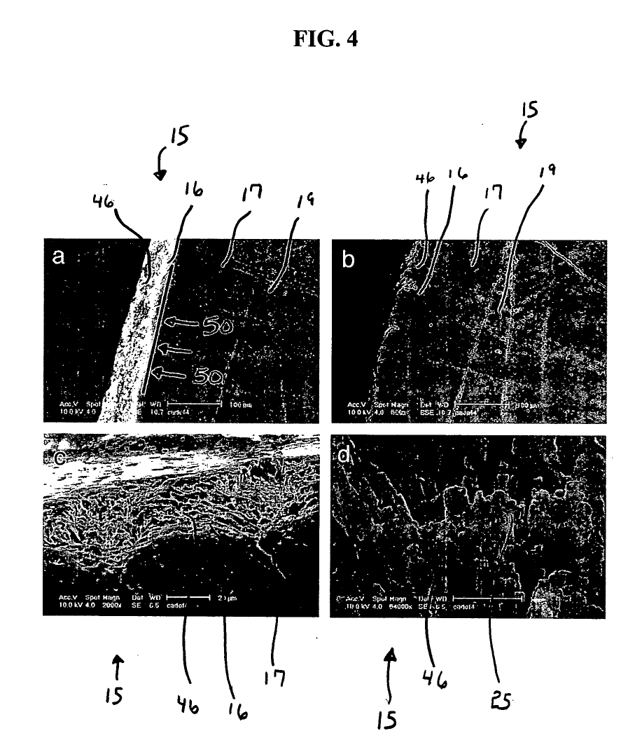

FIG. 4A shows a scanning electron microscopy (SEM) image of a portion of a polished cross section, including the synthetic enamel 46, of the tooth 15 after application of the dental paste 36 containing FHAp of the present invention. In the embodiment shown in FIG. 4A, the synthetic enamel 46, the enamel 16, the dentin 17 and the pulp 19 are shown. After at least one application of the dental paste 36 containing FHAp, the dental paste 36 containing FHAp integrates with the enamel 16 to form the synthetic enamel 46. In the embodiment of the present invention shown in FIG. 4A, the dental paste 36 containing FHAp is strongly integrated with the enamel 16 shown by the lack of contrast between the enamel 16 and the synthetic enamel 46. The transition zone 50 is shown as the area where the synthetic enamel 46 integrates with the enamel 16. The transition zone 50 is shown where the light grey intersects the darker grey.

FIG. 4B shows a back scattered scanning electron microscope image of a portion of the polished cross section, including the synthetic enamel 46, of the tooth 15 after application of the dental paste 36 containing FHAp of the present invention. FIG. 4B is the same tooth 15 and view as FIG. 4A, but with the back scatter technique used to show the absence of a contrast between the synthetic enamel 46 and the enamel 16. In the embodiment of the present invention shown in FIG. 4B, the back scattered scanning electron microscope image shows the portion of the polished cross section of the tooth 15 as if the polished cross section of the tooth 15 was comprised of a single constituent. By showing the polished section of the tooth 15 as a single constituent, the back scattered scanning electron microscope image of FIG. 4B confirms the dental paste 36 containing FHAp is strongly integrated with the enamel 16 to form a strong synthetic enamel 46. In addition, without a contrast between the applied dental paste 36 containing FHAp and the enamel 16, FIG. 4B shows the integrated dental paste 36 containing FHAp has a similar chemical composition as the enamel 16.

FIG. 4C shows a secondary scanning electron microscope image of an inner structure of the portion of the polished tooth 15, including the synthetic enamel 46, after application of the dental paste 36 containing FHAp of the present invention. In the embodiment of the present invention shown in FIG. 4C, the thickness of the synthetic enamel 46 is between about 30 microns and about 50 microns. As discussed previously, various factors including the number of applications of the dental paste 36 containing FHAp to the tooth 15 affect the thickness of the synthetic enamel 46. As shown in FIG. 4C, the dental paste 36 containing FHAp integrates into the enamel 16 producing the synthetic enamel 46, shown as a net like structure.

FIG. 4D shows a high magnification secondary scanning electron microscope image of the synthetic enamel 46 after application of the dental paste 36 containing FHAp of the present invention. As shown in FIG. 4C, the synthetic enamel 46 comprises a plurality of micron-scaled pillar crystals 25. The plurality of pillar crystals 25 are arranged in a direction not parallel (approximately perpendicular) to a surface of the tooth 15. The structure and direction arrangement of the plurality of pillar crystals 25 of the synthetic enamel 46 is approximately the same as the structure and arrangement direction of the enamel rods or prisms of the enamel 16 of the tooth 15.

The integration of the dental paste 36 containing FHAp into the enamel 16 of the tooth 15 fills in a plurality of voids in a lattice of the hydroxyapatite compound comprising the enamel 16. The dental paste 36 containing FHAp integrates with a chemically incomplete portion of the tooth 15 to crystallize the chemically incomplete portion of the tooth 15. The integration of the dental paste 36 containing FHAp results in the pillar crystal 25 morphology shown in FIG. 4D. The plurality of hydroxyapatite crystals of the enamel 16 are products of the integration of the dental paste 36 containing FHAp with the enamel 16. When compared to the chemical composition and the crystal structure of the hydroxyapatite of the enamel 16 of the tooth 15 that has not been treated with the dental paste 36 containing FHAp, the chemical composition and the crystal structure of the synthetic enamel 46 shown in FIG. 4B and FIG. 4D are about the same as the chemical composition and crystal structure of the untreated tooth 15 prior to treatment with the present invention. In addition, the crystallization of the dental paste 36 containing FHAp to the enamel 16 provides a smooth contrast between the dental paste 36 and the enamel 16.

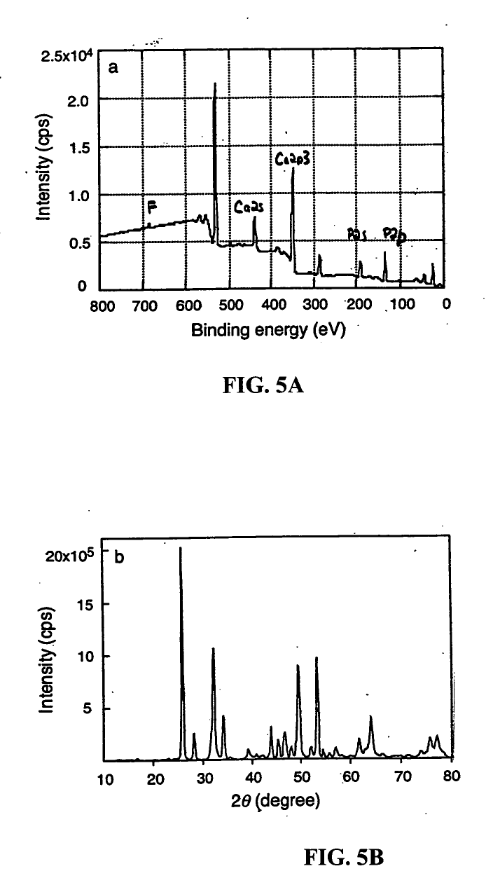

FIG. 5A shows a wide scan x-ray photoelectron spectroscopy (XPS) spectrum of the synthetic enamel 46 of the present invention. FIG. 5A shows a graph of the intensity versus the binding energy. When compared to a XPS spectrum of the enamel 16 of the tooth 15 that has not been treated with the dental paste 36 containing FHAp, the various intensity peaks shown in FIG. 5A corresponding to the synthetic enamel 46 approximately coincide with the peaks of the XPS spectrum of the enamel 16 of the tooth 15 prior to treatment with the present invention. Intensity peaks corresponding to F, Ca2s, Ca2p3, P2s and P2p of the synthetic enamel 46 are shown on FIG. 5A and coincide with F, Ca2s, Ca2p3, P2s and P2p intensity peaks of the enamel 16 of the tooth 15 prior to treatment with the present invention. The synthetic enamel 46 comprises Ca²⁺, P⁵⁺ and F− ions that are fundamentally the same as the Ca²⁺, P⁵⁺ and F⁻ ions in the enamel 16. In addition, the amount of F⁻ ions in the synthetic enamel 46 is about the same as the amount of F− ions in the enamel 16. The Ca/P atomic ratio of the synthetic enamel 46 in the present invention is about 1.58 while a Ca/P atomic ratio of the enamel is about 1.64.

FIG. 5B shows a x-ray diffraction (XRD) pattern of the synthetic enamel 46 of the present invention. When compared to a XRD pattern of the enamel 16 of the tooth 15 prior to treatment with the present invention, the detected peaks shown in FIG. 5B corresponding to the synthetic enamel 46 approximately coincide with the detected peaks of the XRD pattern of the enamel 16 of the tooth 15 prior to treatment with the present invention. Sharp intense 002 reflections indicate that the synthetic enamel 46 consists of single crystals with arranging [0001] direction. The apatite crystal is bounded by the (0001) crystallographic face (also known as the c-face), and the (10\overline{1}0) face (also known as the a face). The (0001) face corresponds to the basal face of the apatite crystal while the (10\overline{1}0) face corresponds to the sides of the crystal. The single crystals arranged in the [0001] direction are in a direction approximately perpendicular to the (0001) face.

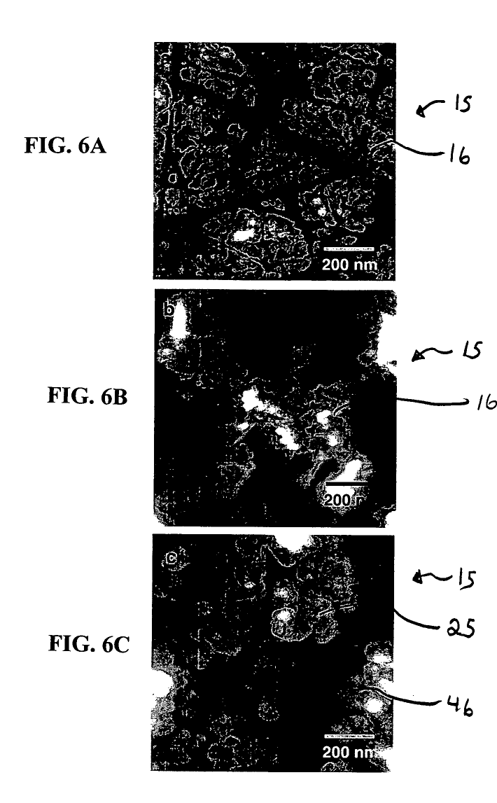

FIGS. 6A-6C show the integration of the dental paste 36 containing FHAp of the present invention with the enamel 16 and how the morphology of the fluoride treated hydroxyapatite crystals are extended to [0001] direction. FIG. 6A shows an atomic force microscopy (AFM) image of a polished surface of the enamel 16 of the tooth 15 before treatment with the dental paste 36 containing FHAp of the present invention. FIG. 6A illustrates a plurality of stretched lines resulting from the polishing of the surface. FIG. 6B shows the AFM image of the polished surface of the tooth 15 after application of the dental paste 36 containing FHAp to the enamel 16 for about one minute and a subsequent rinse of the dental paste 36 containing FHAp with a washing medium from the tooth 15. FIG. 6B shows an intermediate step where the dental paste 36 containing FHAp dissolves a portion of the enamel 16 and the dental paste 36 containing FHAp is partially integrated into the enamel 16. FIG. 6C shows the AFM image of the polished surface of the tooth 15 after about 15 minutes of treatment and a subsequent rinse of the dental paste 36 containing FHAp with the washing medium from the tooth 15. As shown in FIG. 6C, the surface of the tooth 15 is covered with particles having diameters between about 30 nanometers (nm) and about 50 nm. Combined with the SEM images of FIGS. 4C-4D, FIG. 6C shows that the particles are the top part of the pillar crystals 25 shown in FIG. 4D.

In another embodiment of the present invention, a dental paste 38 containing SiHAp is applied to the tooth 15 either alone or in addition to the dental paste 36 containing FHAp to enhance the prevention and the treatment of the dental carie 44. In one embodiment of the present invention, the dental paste 38 containing SiHAp is applied after application of the dental paste 36 containing FHAp. In another embodiment of the present invention, the dental paste 38 containing SiHAp is applied before application of the dental paste 36 containing FHAp. The dental paste 38 containing SiHAp can enhance the treatment of the dental carie 44 and help promote the replacement of the dental carie 44. The SiHAp powder may be applied to the tooth either before or after application of the dental paste 36 containing FHAp. By applying SiHAp powder to the tooth after application of the dental paste 36 containing FHAp, the thickness of the synthetic enamel is increased and the appearance of the tooth is improved due to a greater luster, shine, and polishability. Those skilled in the art will recognize that any number of applications of either or both of the dental pastes to the tooth are within the spirit and scope of the present invention. Those skilled in the art will also recognize that the dental pastes can be removed from the tooth 15 in many ways known in the art and be within the spirit and scope of the present invention.

Preparation of the dental paste 38 containing SiHAp consists of several steps. In the process of the formation of the silicate containing hydroxyapatite (SiHAp) powder, samples are synthesized by using a wet process. A mixed solution of phosphoric acid-sodium silicate (both available from Kanto Pure Chemicals Co. Ltd., Japan) is prepared having a total concentration of PO₄³⁻ and SiO₄⁴⁻ of about 0.3 mol.dm⁻³ and added to a calcium hydroxide Ca(OH)₂ slurry containing about 0.5 mol.dm⁻³ of Ca²⁺ ion (available from UBE Material, Ltd., Japan). The sodium silicate decreases the viscosity of the silicate containing hydroxyapatite paste. The compositions of the samples can be controlled by changing synthesis temperature, and contents of trace elements are controlled by selecting the purity of Ca(OH)2. Furthermore, the molar ratio of phosphate ion and silicate ion (Si/P ratio) in samples are controlled by selecting the Si/P ratio in mixed solution of phosphoric acid-sodium silicate. The molar ratio of Si/P may range from about 0.11 to about 0.43. The synthesis temperature may range from about 5 to 40° C. A method of forming the silicate containing hydroxyapatite powder is known in the art, such as for example, the silicate containing hydroxyapatite powder taught in Okada and Suzuki, 2001, Journal of the Society of Inorganic Materials, Japan, 8: 118-124, the entirety of which is hereby incorporated by reference. About 2 ml of a mix of an about 35% aqueous solution of hydrogen peroxide and an about 85% aqueous solution of phosphoric acid is added to and mixed with about 1.5 g of the mixture of the phosphoric acid, the sodium silicate and the calcium hydroxide suspension to yield the dental paste 38 containing SiHAp of the present invention. Those skilled in the art will recognize that various aqueous solutions and powders known in the art can be used with the present invention and other amounts and concentrations of the aqueous solutions and powders in addition to other processing conditions known in the art are within the spirit and scope of the present invention.

After the dental paste 38 containing SiHAp is mixed, the dental paste 38 containing SiHAp is applied to the tooth 15 where the dental paste 38 containing SiHAp integrates with the enamel 16. The extent of integration is governed by the depth of the natural enamel 16 of the tooth 15. In integrating with the enamel 16, the dental paste 38 containing SiHAp fills in a plurality of voids in a lattice of the hydroxyapatite compound comprising the enamel 16. The dental paste 38 containing SiHAp crystallizes with a chemically incomplete portion of the enamel 16 of the tooth, forming silicate apatite crystals of the synthetic enamel 46. In a preferred embodiment of the present invention, the ingredients used to make the dental paste 38 containing SiHAp are mixed prior to use, as the dental paste 38 containing SiHAp will undergo gradual loss of activity. About 10 minutes after application of the dental paste 38 containing SiHAp to the enamel 16, the excess dental paste 38 containing SiHAp that has not integrated with the enamel 16 is removed by washing the tooth 15 with a washing medium. In a preferred embodiment of the present invention, the washing medium is purified water. Those skilled in the art will recognize that the dental paste can remain on the tooth for various lengths of time and be within the spirit and scope of the present invention.

In a preferred embodiment of the present invention, the dental paste 38 containing SiHAp is applied to the enamel 16 of the tooth 15 several times. In a preferred embodiment of the present invention, the dental paste 38 containing SiHAp is applied to the enamel 16 of the tooth 15 from about two times to about six times. In another preferred embodiment of the present invention, the dental paste 38 containing SiHAp is applied to the enamel 16 of the tooth 15 about three times. Each application of the dental paste 38 containing SiHAp to the tooth 15 increases the thickness of the synthetic enamel 46, allowing for additional integration of the dental paste 38 containing SiHAp into the enamel 16 of the tooth 15. After each application of the dental paste 38 containing SiHAp to the tooth 15, the excess dental paste 38 containing SiHAp is removed by washing the tooth 15 with the washing medium. In an alternative embodiment of the present invention, a high and/or low speed handpiece is used in addition to the application of the dental paste 38 containing SiHAp. Those skilled in the art will recognize that any number of applications of the dental paste to the tooth are within the spirit and scope of the present invention. Those skilled in the art will also recognize that the dental paste 38 containing SiHAp can be removed from the tooth 15 in many ways known in the art and be within the spirit and scope of the present invention.

For a tooth showing a susceptibility to the formation of a dental carie 44, the dental paste 38 containing SiHAp integrates with the incomplete enamel structure to form the synthetic enamel 46. The dental paste 38 containing SiHAp mechanically and chemically completes the enamel structure of the tooth 15, completing the apatite of the enamel 16 to prevent the dental carie 44 from forming.

The dental paste 38 containing SiHAp of the present invention treats the dental carie 44 and replaces the dental carie 44 with the synthetic enamel 46. Those skilled in the art will recognize the dental paste 38 containing SiHAp can be used to treat dental caries 44 in various stages ranging from a stage where there is no dental carie 44 to stages where the dental carie 44 is deep within the tooth 15 and be within the spirit and scope of the present invention.

For the tooth having a dental carie 44, the dental paste 38 containing SiHAp is used to treat the dental carie 44 existing in the enamel 16 of the tooth 15. In another embodiment of the present invention, the dental paste 38 containing SiHAp is used to treat the dental carie 44 that has advanced into the dentin 17 of the tooth 15. In another embodiment of the present invention, the dental paste 38 containing SiHAp can be used as a cement. Those skilled in the art will recognize the dental paste 38 containing SiHAp can be used to treat dental caries 44 that have progressed into various portions of the tooth 15 and are within the spirit and scope of the present invention.

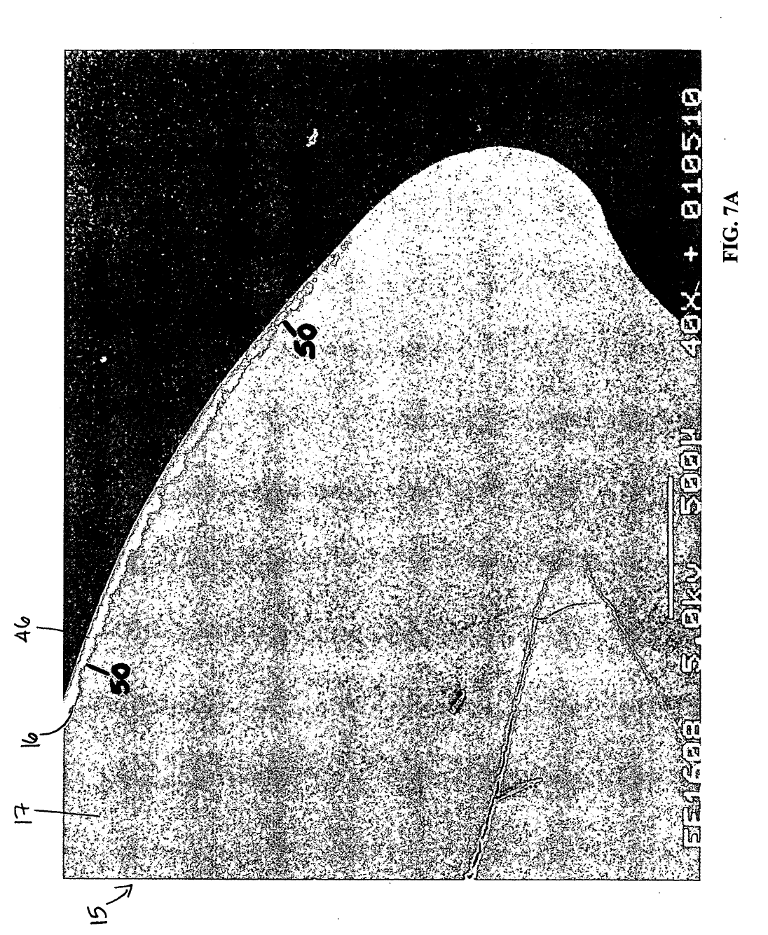

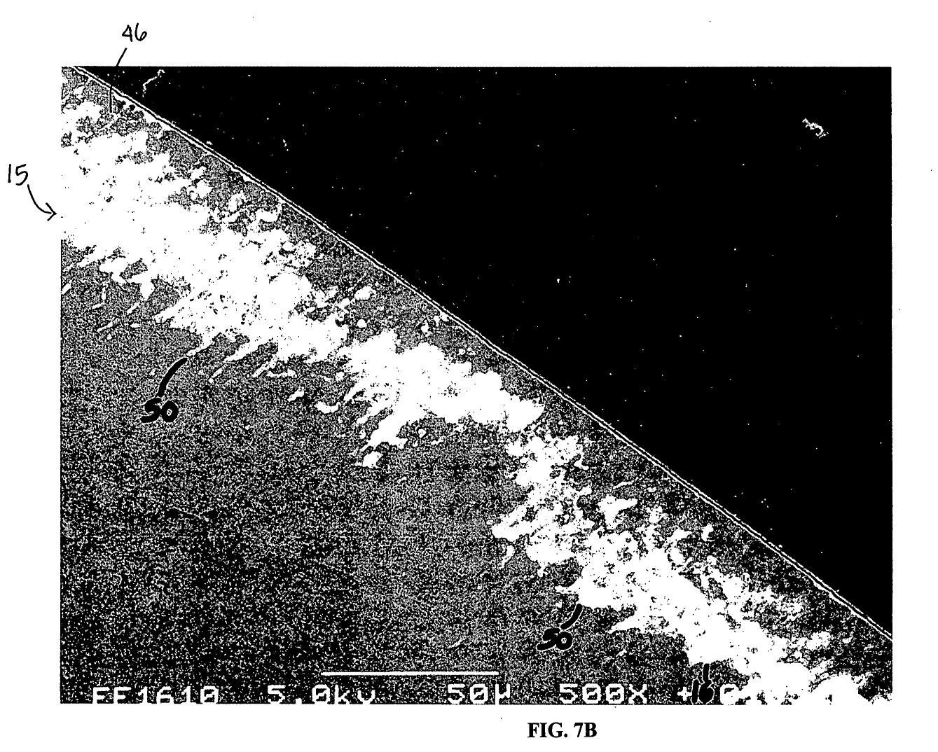

FIG. 7A shows a low magnification (40×) scanning electron microscopy image of a portion of a polished cross section, including the synthetic enamel 46, of the tooth 15 after application of the dental paste 38 containing SiHAp of the present invention. In the embodiment shown in FIG. 7A, the synthetic enamel 46, the enamel 16 and the dentin 17 are shown. After at least one application of the dental paste 38 containing SiHAp, the dental paste 38 containing SiHAp integrates with the enamel 16 to form the synthetic enamel 46. The integration of the dental paste 38 containing SiHAp into the enamel 16 of the tooth 15 fills in a plurality of voids in a lattice of the hydroxyapatite compound comprising the enamel 16. The dental paste 38 containing SiHAp integrates with a chemically incomplete portion of the tooth 15 to crystallize the chemically incomplete portion of the tooth 15. The transition zone 50 is shown as the area where the synthetic enamel 46 integrates with the enamel 16. The transition zone 50 is shown where the light grey intersects the darker grey.

FIG. 7B shows a high magnification (500×) scanning electron microscope image of the synthetic enamel 46 after application of the dental paste 38 containing SiHAp of the present invention. The integration of the dental paste 38 containing SiHAp results in a pillar crystal 25 morphology. The plurality of hydroxyapatite crystals of the enamel 16 are products of the integration of the dental paste 38 containing SiHAp with the enamel 16. The crystallization of the dental paste 38 containing SiHAp to the enamel 16 provides a smooth contrast between the dental paste 38 containing SiHAp and the enamel 16. The transition zone 50 is shown as the area where the synthetic enamel 46 integrates with the enamel 16. The transition zone 50 is shown where the light grey intersects the darker grey.

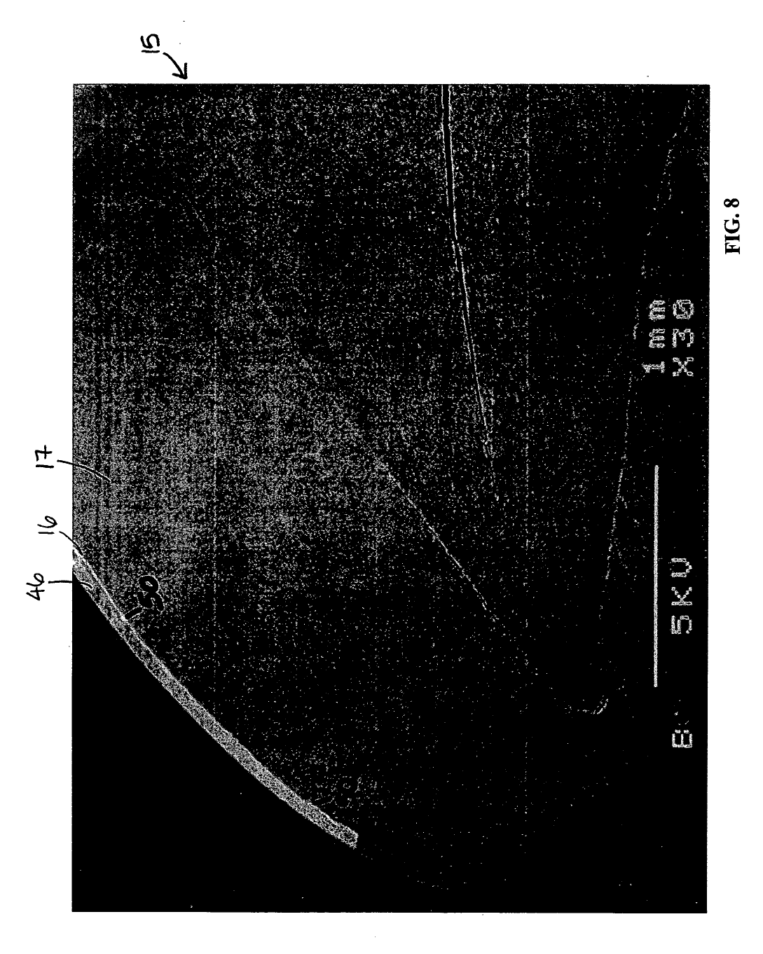

FIG. 8 shows a scanning electron microscopy image of a portion of a polished cross section, including the synthetic enamel 46, of the tooth 15 after application of the dental paste 36 containing FHAp of the present invention. In the embodiment shown in FIG. 8, the synthetic enamel 46, the enamel 16 and the dentin 17 are shown. After at least one application of the dental paste 36 containing FHAp, the dental paste 36 containing FHAp integrates with the enamel 16 to form the synthetic enamel 46. In the embodiment of the present invention shown in FIG. 8, the dental paste 36 containing FHAp is strongly integrated with the enamel 16 shown by the lack of contrast between the enamel 16 and the synthetic enamel 46. The transition zone 50 is shown as the area where the synthetic enamel 46 integrates with the enamel 16. The transition zone 50 is shown where the light grey intersects the darker grey.

When comparing FIGS. 7A and 7B with FIG. 8, it is evident that the application of dental paste 38 containing SiHAp to a tooth 15 results in an increase in the crystallization of the synthetic enamel 46 as compared with application of the dental paste 36 containing FHAp.

Without being limited to any particular theory, the increase in the crystallization of the synthetic enamel 46 after application of the dental paste 38 containing SiHAp is partly due to the larger size of the SiHAp molecules as compared to the FHAp molecules. The larger size of the SiHAp molecules also results in an increase in the thickness of the dental paste 38 containing SiHAp as compared to the dental paste 36 containing FHAp for the same number of applications. Thus, if fewer layers of dental paste are desired, one should use the dental paste 38 containing SiHAp. The dental paste 38 containing SiHAp provides, among other things, better polishability (i.e., better surface finish after polishing with dental tools) and an increase in viscosity (which affects the wettability of the dental paste) as compared with the dental paste 36 containing FHAp.

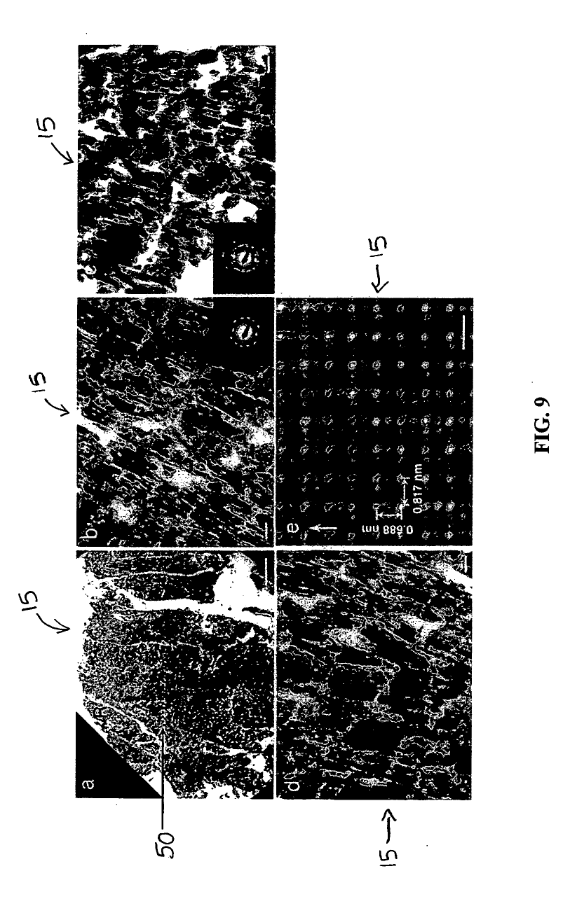

FIGS. 9A-E show transmission electron microscopy images of a thin tooth section after application of the dental paste 36 containing FHAp of the present invention. FIG. 9A is a low magnification of the tooth 15 including the synthetic enamel 46 (labeled as R in FIG. 9A) and the enamel 16 (labeled as E in FIG. 9A). The transition zone 50 is shown as the arrow and dotted line and is the transition between the synthetic enamel 46 and the enamel 16 (scale bar is 5 μm). FIG. 9B shows a magnified image of the synthetic enamel 46 of the tooth 15. The synthetic enamel 46 consists of elongated pillar crystals 25 arranged in the same orientation (scale bar is 100 nm). An electron diffraction pattern of the observed area is superimposed at the bottom right corner of FIG. 9B. FIG. 9C shows a magnified image of the enamel 16 region (scale bar is 100 nm). An electron diffraction pattern of the observed area is superimposed at the bottom left corner of FIG. 9C.

FIG. 9D is a magnified image around the interface between the synthetic enamel 46 and the enamel 16 region of the tooth 15. As shown in FIG. 9D, pillar crystals 25 grow continuously, i.e., there is no discontinuous boundary observed (scale bar is 100 nm). Continuous growth of pillar crystals leads to better integration of the synthetic enamel 46 and the enamel 16. FIG. 9E is a high-resolution image showing the crystal structure of the synthetic enamel 46 of the tooth 15 (scale bar is 1 nm). Crystal lattice periodicities on the nanometer scale were observed as shown in FIG. 9E (0.817 nm and 0.688 nm). The arrows in FIG. 9E indicate the elongated direction of the crystal.

Those skilled in the art will recognize that apatite powders other then fluorized apatite (FHAp) and silicate containing hydroxyapatite (SiHAp) can be used in the compositions and methods of the present invention. The general chemical formula for the apatite used in the compositions of the present invention is A₁₀(BO₄)₆C₂, where A is calcium (Ca), B is phosphate (P) and C is fluorine, chlorine or hydroxyl (F, Cl, OH). The F, Cl and OH ions can freely substitute in the crystal lattice of apatite and all three are usually present in every specimen although some specimens have been close to 100% in one or the other. For example, the fluorized apatite (FHAp) powder used in the dental paste of the present invention has a chemical formula of Ca₁₀(PO₄)₆F₂, in which the C is fluorine. The silicate containing hydroxyapatite (SiHAp) powder used in the dental paste of the present invention has a chemical formula of Ca₁₀(POSi)₆OH₂, in which the C is hydroxyl and the PO₄³⁻ ion in the hydroxyapatite has been partially substituted for SiO₄⁴⁻ ion.

In three-dimensional space, fourteen different lattices divided among six crystal systems may be constructed. These are the isometric or cubic, tetragonal, orthorhombic, monoclinic, triclinic, and hexagonal systems, as established by French crystallographer Auguste Bravais. The apatite mineral belongs to the hexagonal crystal system of the Bravais lattice. Minerals of the hexagonal crystal system are referred to three crystallographic axes which intersect at 120° and a fourth which is perpendicular to the other three. This fourth axis is usually depicted vertically. All crystals of the hexagonal division possess a single 6-fold axis of rotation. In addition to the single 6-fold axis of rotation, crystals of the hexagonal division may possess up to six 2-fold axes of rotation. Crystals of the hexagonal division may demonstrate a center of inversion symmetry and up to seven mirror planes. Those skilled in the art will recognize that any apatite powder that meets the above criteria can be used in the compositions and methods with the spirit and scope of the present invention.

In other embodiments of the present invention, the dental paste compositions of the present invention can be used in various fields both inside and outside of the field of dentistry. For example, the dental paste compositions can be used in the field of orthodontics, oral surgery, periodontics, endodontics, operative/restorative dentistry, prosthodontic dentistry, preventive dentistry, implantology, column chromatography, piezoelectric materials and semiconductors.

In the field of orthodontics, the dental paste compositions reduce the need for strategic tooth extractions, such as the extraction of premolars in order to create space to mobilize teeth. By combining the enamel plasty technique, a technique that usually removes enamel from the mesial and distal sides of the tooth, and the application, building and contouring of the dental paste compositions, the orthodontist is able to control the space needed for orthodontic treatment without teeth extraction.

In the field of oral surgery and periodontics, the dental paste compositions can be used to treat a site of alveolar bone loss or deficiency. For example, the fluoride treated calcium deficient hydroxyapatite powder dental paste composition of the present invention is mixed in succinic acid solution or a similar solution and applied to fill the void from the bone loss or deficiency as a bone cement or artificial bone.

In addition to its use for filling sites of bone loss in the fields of oral surgery and periodontics, the fluoride treated calcium deficient hydroxyapatite powder dental paste composition of the present invention can be used in operative/restorative dentistry and prosthodontic dentistry to protect the pulp, prevent further tooth decay and reduce the sensitivity which a patient experiences. When the pulp chamber is close to being exposed after excavation of the dental caries and preparation of the restoration, the solidified and shaped fluoride treated calcium deficient hydroxyapatite powder dental paste composition can be placed instead of the base material to protect the pulp, prevent further tooth decay and reduce the sensitivity. The dental paste compositions also transition unsupported enamel into a supported enamel by integrating into the tooth to provide a synthetic enamel, thereby making restorations more conservative.

The dental paste compositions have use in the field of operative dentistry and prosthodontic dentistry. In these fields, the dental paste compositions can be used as a restorative material in place of composite resins or cement materials.

Further enhancement of the prevention of dental caries in the field of preventive dentistry can be achieved by mixing the dental paste compositions of the present invention with toothpaste, prophy paste and fluoride treatment gel. The dental paste compositions of the present invention are more effective than prior art dental sealants that stay in place due to adhesion, rather than integration to the tooth structure.

The dental paste compositions can be used in the field of implantology as well. For applications in this field, the dental paste compositions are applied to a metal base of the implant and used as an artificial calcium of the natural tooth to coat the base metal.

When periodontitis, gingivitis and/or other factors cause an exposure of the cementum of the tooth and a subsequent sensitivity due to the exposed cementum occurs, the dental paste compositions can be used as a coating agent on the exposed cementum to reduce the sensitivity. Furthermore, the dental paste compositions, mixed with polysaccharide (especially maltotriose) and/or soy bean fiber powder, can be applied to the cementum to act as an artificial cementum.

The dental paste compositions of the present invention are also useful as disinfectants or antibacterial agents. The disinfectant effect or antibacterial effect helps prevent the formation of dental caries. The dental paste compositions can be applied to a tooth with or without a dental carie.

Various applications of the dental paste compositions outside of the field of dentistry include the use of the dental paste compositions as a filler material for column chromatography, piezoelectric materials or surface application materials of semiconductors. For these uses, for example, the fluoride treated calcium deficient hydroxyapatite dental paste composition is ionized in the low pH solution and then recrystallized. This produces a more dense, regular, larger and higher quality of the crystal when compared to the case where the naturally occurring calcium phosphate cluster is ionized in the same low pH solution and recrystallized. The higher quality of the crystal allows use in the aforementioned fields outside of the field of dentistry.

In an embodiment, the present invention provides a dental paste to treat a dental carie 44 of a tooth 15 including a fluorized apatite powder; an aqueous solution of hydrogen peroxide; and an aqueous solution of phosphoric acid.

In an embodiment, the present invention provides a method of treating a dental carie 44 of a tooth 15 including applying a dental paste comprising a fluorized apatite (FHAp) powder mixed with an aqueous solution of hydrogen peroxide and an aqueous solution of phosphoric acid to the tooth 15 having the dental carie 44 and washing an excess amount of the dental paste 36 containing FHAp from the tooth 15. The process is repeated until the dental carie 44 of the tooth 15 is treated. The method may further include applying a dental paste 38 containing SiHAp to the tooth 15 having the dental carie 44 and washing the excess dental paste 38 containing SiHAp from the tooth 15.

In an embodiment, the present invention provides a method of preventing formation of a dental carie 44 of a tooth 15 including: (a) applying a dental paste 36 comprising a fluorized apatite (FHAp) powder mixed with an aqueous solution of hydrogen peroxide and an aqueous solution of phosphoric acid to the tooth 15 and (b) washing an excess amount of the dental paste 36 containing FHAp from the tooth 15.

In an embodiment, the present invention provides a method of making a composition to prevent and treat a dental carie 44 including: mixing an aqueous solution of sodium fluoride and a calcium phosphate; filtering a precipitate of a mixture of the aqueous solution of sodium fluoride and the calcium phosphate; reducing the precipitate to a plurality of small particles and passing the plurality of small particles through a sleeve to yield a fluorized apatite powder; and mixing the fluorized apatite powder with an aqueous solution of hydrogen peroxide and an aqueous solution of phosphoric acid.

In an embodiment, the present invention provides a method of whitening a tooth including: applying a dental paste 36 comprising a fluorized apatite (FHAp) powder mixed with an aqueous solution of hydrogen peroxide and an aqueous solution of phosphoric acid to the tooth 15 and washing an excess amount of the dental paste 36 containing FHAp from the tooth 15.

In an embodiment, the present invention provides a dental paste to treat a dental carie 44 of a tooth 15 including a silicate containing hydroxyapatite powder; an aqueous solution of hydrogen peroxide; and an aqueous solution of phosphoric acid.

In an embodiment, the present invention provides a method of treating a dental carie 44 of a tooth 15 including: (a) applying a dental paste 38 comprising a silicate containing hydroxyapatite (SiHAp) powder mixed with an aqueous solution of hydrogen peroxide and an aqueous solution of phosphoric acid to the tooth 15 having the dental carie 44 and (b) washing an excess amount of the dental paste 38 containing SiHAp from the tooth 15.

In an embodiment, the present invention provides a method of preventing formation of a dental carie 44 including: (a) applying a dental paste 38 comprising a silicate containing hydroxyapatite (SiHAp) powder mixed with an aqueous solution of hydrogen peroxide and an aqueous solution of phosphoric acid to the tooth 15 and (b) washing the excess dental paste 38 containing SiHAp from the tooth 15.

In an embodiment, the present invention also provides a method of making a dental paste 38 containing SiHAp to prevent and treat a dental carie 44 including: mixing an aqueous solution of phosphoric acid-sodium silicate and a calcium phosphate; filtering a precipitate of a mixture of the aqueous solution of phosphoric acid-sodium silicate and the calcium phosphate; reducing the precipitate to a plurality of small particles and passing the plurality of small particles through a sleeve to yield a silicate containing hydroxyapatite powder; and mixing the silicate containing hydroxyapatite powder with an aqueous solution of hydrogen peroxide and an aqueous solution of phosphoric acid.

Further, the present invention provides a method of whitening a tooth 15 including: applying a dental paste 38 comprising a silicate containing hydroxyapatite (SiHAp) powder mixed with an aqueous solution of hydrogen peroxide and an aqueous solution of phosphoric acid to the tooth 15 and washing the excess dental paste 38 containing SiHAp from the tooth 15.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. A dental paste to treat a dental carie of a tooth comprising:
a silicate containing hydroxyapatite powder;
an aqueous solution of hydrogen peroxide; and
an aqueous solution of phosphoric acid.

2. The dental paste of claim 1 wherein the silicate containing hydroxyapatite powder is comprised of a calcium phosphate and an aqueous solution of phosphoric acid-sodium silicate.

3. The dental paste of claim 2 wherein the calcium phosphate is a hydroxyapatite.

4. The dental paste of claim 1 wherein the aqueous solution of phosphoric acid is selected from the group consisting of metaphosphoric acid, pyrophosphoric acid, ortho-phosphoric acid and triphosphate.

5. The dental paste of claim 1 wherein the dental paste integrates with an enamel of the tooth having the dental carie to form a synthetic enamel of the tooth.

6. The dental paste of claim 1 wherein the dental paste crystallizes with an enamel of the tooth having the dental carie by integrating with a chemically incomplete portion of the enamel of the tooth.

7. The dental paste of claim 1 wherein the dental paste fills in a plurality of voids in a lattice of a hydroxyapatite compound comprising an enamel of the tooth.

8. A method of treating a dental carie of a tooth comprising:
(a) applying a dental paste comprising a silicate containing hydroxyapatite powder mixed with an aqueous solution of hydrogen peroxide and an aqueous solution of phosphoric acid to the tooth having the dental carie; and
(b) washing an excess amount of the dental paste from the tooth.

9. The method of claim 8 further comprising repeating steps (a) and (b) until the dental carie is treated.

10. The method of claim 8 further comprising applying a second dental paste comprising a fluorized apatite powder mixed with an aqueous solution of hydrogen peroxide and an aqueous solution of phosphoric acid to the tooth having the dental carie and washing an excess amount of the second dental paste from the tooth until the dental carie is treated.

11. The method of claim 8 further comprising crystallizing the dental paste in the tooth.

12. The method of claim 8 further comprising replacing the dental carie with a plurality of crystals from the dental paste.

13. The method of claim 8 further comprising integrating the dental paste into an enamel of the tooth to form a synthetic enamel comprised of the dental paste.

14. The method of claim 13 wherein the synthetic enamel has a crystal structure and chemical composition approximately the same as a hydroxyapatite of the enamel.

15. The method of claim 13 wherein the dental paste integrates with a chemically incomplete portion of the enamel of the tooth to crystallize a chemically incomplete portion of the enamel of the tooth.

16. A method of preventing formation of a dental carie comprising:
(a) applying a dental paste comprising a silicate containing hydroxyapatite powder mixed with an aqueous solution of hydrogen peroxide and an aqueous solution of phosphoric acid to the tooth; and
(b) washing an excess amount of the dental paste from the tooth.

17. The method of claim 16 further comprising applying a second dental paste comprising a fluorized apatite powder mixed with an aqueous solution of hydrogen peroxide and an aqueous solution of phosphoric acid to the tooth and washing an excess amount of the second dental paste from the tooth.

18. A method of making a dental paste to prevent and treat a dental carie comprising:
mixing an aqueous solution of phosphoric acid-sodium silicate and a calcium phosphate;
filtering a precipitate of a mixture of the aqueous solution of phosphoric acid-sodium silicate and the calcium phosphate;
reducing the precipitate to a plurality of small particles and passing the plurality of small particles through a sleeve to yield a silicate containing hydroxyapatite powder; and
mixing the silicate containing hydroxyapatite powder with an aqueous solution of hydrogen peroxide and an aqueous solution of phosphoric acid.

19. A method of whitening a tooth comprising:
applying a dental paste comprising a silicate containing hydroxyapatite powder mixed with an aqueous solution of hydrogen peroxide and an aqueous solution of phosphoric acid to the tooth; and
washing an excess amount of the dental paste from the tooth.

20. The method of claim 19 further comprising applying a second dental paste comprising a fluorized apatite powder mixed with an aqueous solution of hydrogen peroxide and an aqueous solution of phosphoric acid to the tooth and washing an excess amount of the second dental paste from the tooth.
